(19) United States
(12) Patent Application Publication
(10) Pub. No.: US 2026/0086094 A1
(43) Pub. Date: Mar. 26, 2026

(54) BIOMARKER PANEL, MICROFLUIDIC DEVICE AND DETECTION KIT FOR CAPTURING CIRCULATING TUMOR CELLS

(71) Applicant: WEST CHINA HOSPITAL OF SICHUAN UNIVERSITY, chengdu (CN)

(72) Inventors: Xiaowei LIU, Chengdu (CN); Leyi TANG, Chengdu (CN); Hubing SHI, Chengdu (CN); JING Jing, Chengdu (CN); Jinen SONG, Chengdu (CN)

(21) Appl. No.: 19/223,569

(22) Filed: May 30, 2025

(30) Foreign Application Priority Data
Sep. 26, 2024 (CN) 202411351062.7

(51) Int. Cl. G01N 33/574; B01L 3/00; G01N 33/543; G01N 33/68

(52) U.S. Cl. CPC G01N 33/57525; B01L 3/502753; G01N 33/54386; G01N 33/57515; G01N 33/6854; G01N 33/6872; B01L 2300/041; B01L 2300/047; B01L 2300/1894; B01L 2400/0487; G01N 2333/4704; G01N 2333/70557; G01N 2333/70596

(57) ABSTRACT

The present disclosure belongs to the field of tumor diagnosis, and specifically relates to a biomarker panel, a microfluidic device and a detection kit for capturing circulating tumor cells. The biomarker panel includes a first biomarker and a second biomarker; wherein: the first biomarker is EPCAM; the second biomarker is one or a combination of two or more of CD9, CD41, THBS1, RGS18, and RGS10; the circulating tumor cells are pancreatic cancer circulating tumor cells and/or breast cancer circulating tumor cells. In the present disclosure, by using antibodies against novel surface biomarkers of pancreatic cancer or breast cancer circulating tumor cells (CTCs), the enrichment and detection rate of CTCs in the blood of patients are significantly improved, and the possibility of missed detection is effectively reduced; meanwhile, the capture and enrichment efficiency of CTCs are enhanced, thus providing strong support for dynamic tumor monitoring, prognostic evaluation and personalized precise treatment of cancer patients, and offering broad market value and application prospects.

### CROSS REFERENCE

This application claims priority to Chinese application No. 202411351062.7 under 35 US Code Section 119 (e). The Chinese application was filed on Sep. 26, 2024, titled “BIOMARKER PANEL, MICROFLUIDIC DEVICE AND DETECTION KIT FOR CAPTURING CIRCULATING TUMOR CELLS”, the entire content of which is hereby incorporated by reference into this application.

### TECHNICAL FIELD

The present disclosure belongs to the field of tumor diagnosis, and specifically relates to a biomarker panel, a microfluidic device and a detection kit for capturing circulating tumor cells.

### BACKGROUND

Circulating tumor cells (CTCs) are cancer cells released into the blood circulation by primary tumors or metastatic lesions and are considered to be the “seeds” of cancer metastasis. The presence and number of CTCs are closely related to the patient's risk of metastasis, treatment response, and survival rate. Therefore, the capture and analysis of CTCs can provide critical information for early diagnosis, prognostic prediction and personalized treatment of cancers. However, since CTCs are extremely rare in peripheral blood, with approximately only 1 to 10 CTCs per 1 billion blood cells, their detection and isolation have always been technical challenges in tumor research and clinical applications. Existing CTC detection technologies mainly include immunological methods, isolation methods based on physical properties (e.g., density gradient centrifugation) and microfluidic techniques. Current microfluidic techniques have shown great potential for CTC capture. Microfluidic chips can achieve efficient enrichment and capture of CTCs by precisely controlling hydrodynamic conditions and taking advantage of cell size differences. However, due to the uneven size of circulating tumor cells and the extremely low proportion of cells, the isolation mode of microfluidic techniques lacks specificity. With the development of CTC detection technology, the advantages of immunoscreening are gradually being reflected. Immunoassay for CTC detection mainly relies on the high-affinity binding between specific biomarkers on the surface of tumor cells and antibodies. By combining tumor-related antibodies (e.g., antibodies against EPCAM, CK, HER2, etc.) with detection equipment, CTCs can be specifically captured or labeled. Immunoassays are subdivided into two categories: immunoenrichment and immunostaining. Immunoenrichment is to enrich CTCs in the blood by combining antibodies with specific biomarkers on the surface of CTCs, while immunostaining is to label CTCs with antibodies and then detect them using a flow cytometer or a fluorescence microscope.

Currently, the mainstream methods for CTC capture rely on tumor cell-specific surface biomarkers, such as epithelial cell adhesion molecules (EPCAMs). Many commercially available devices, such as the CellSearch system, enrich CTCs based on EPCAM antibodies and have been widely applied for CTC detection in a variety of cancers. However, the expression of EPCAMs is not high or heterogeneous in some tumor types, resulting in some CTCs not being captured efficiently. In addition, cancer cells may undergo epithelial-mesenchymal transition (EMT) during metastasis, during which the expression of EPCAMs may decrease, further reducing the effectiveness of EPCAM-based capture methods. The use of such a single biomarker for enrichment and screening of CTCs will inevitably result in a certain degree of missed detection. Therefore, identifying new surface biomarkers of tumor cells and developing new CTC capture devices for circulating tumor cells will greatly promote the development of CTC detection technologies.

Therefore, based on this, the technical solution of the present disclosure is proposed.

### SUMMARY

In view of the shortcomings of existing CTC detection technologies, the present disclosure summarizes a series of new surface biomarkers unique to CTCs by performing single-cell sequencing on CTCs and WBCs from pancreatic cancer and breast cancer patients and using bioinformatics techniques to perform big data analysis on the sequencing results.

Furthermore, by comparing the results of population sequencing of protein expression profiles and CTC expression profiles from numerous pancreatic cancer and breast cancer patients, common CTC surface-specific biomarkers were identified. On this basis, enrichment (capture) tests were conducted on pancreatic cancer cell lines and pancreatic cancer patient blood using the newly identified biomarkers to select surface biomarkers with enrichment capability and optimize the optimal combination of new capture antibodies for immuno-enrichment screening. The antibodies or antibody combinations of new surface biomarkers screened by the above method greatly improve the capture and enrichment efficiency of CTCs, which can provide support for clinical evaluation of treatment effects and realization of personalized precise treatment, offering huge market value and application prospects.

A relevant product is used to detect the level of a cell surface biomarker gene expression product in the sample. If the detected level of the cell surface biomarker gene expression product is higher than the reference level, the presence of pancreatic cancer or breast cancer circulating tumor cells (CTCs) is determined.

In order to solve the problems existing in the prior art, the present disclosure provides a biomarker panel for capturing circulating tumor cells, and the biomarker panel comprises a first biomarker and a second biomarker; wherein:
the first biomarker is EPCAM;
the second biomarker is one or a combination of two or more of CD9, CD41, THBS1, RGS18, and RGS10;
the circulating tumor cells are pancreatic cancer circulating tumor cells; and/or
the circulating tumor cells are breast cancer circulating tumor cells.

Based on the same technical concept, another embodiment of the present disclosure is to provide use of a biomarker panel in the preparation of a detection kit for capturing circulating tumor cells.

Based on the same technical concept, another embodiment of the present disclosure is to provide a detection kit comprising a detection reagent for detecting a biomarker panel.

Preferably, the detection kit further comprises detection antibodies for corresponding biomarkers, a microfluidic chip, a buffer, and a flushing fluid; wherein:
the detection antibodies are antibodies against CD9, CD41, THBS1, RGS18, RGS10, and EPCAM;
the microfluidic chip consists of a substrate layer containing multiple channels and a cover layer;
the buffer is a balanced salt solution containing 0.1 wt % BSA and 5 wt % glucose components; and
the flushing fluid is a balanced salt solution containing 0.1 wt % BSA, 0.2 wt % pancreatin, and 0.5 mg/mL papain.

Based on the same technical concept, another embodiment of the present disclosure is to provide use of a biomarker panel in the preparation of a device for capturing circulating tumor cells.

Based on the same technical concept, another embodiment of the present disclosure is to provide a device comprising a microfluidic chip, an injection pump, a connecting pipe, a pre-cooling ice table, and a sample collection tube; wherein:
the microfluidic chip is used to detect the biomarker panel on the surface of circulating tumor cells in a sample and capture the biomarker panel;
the injection pump is used to inject the sample into the microfluidic chip;
the connecting pipe is used to connect the injection pump, the microfluidic chip and the sample collection tube;
the pre-cooling ice table is used to keep the microfluidic chip at low temperature; and
the sample collection tube is used to collect the captured CTCs.

Preferably, the sample is a human blood sample.

The present disclosure also provides a method for detecting or capturing circulating tumor cells (CTCs) in the blood sample of pancreatic cancer and/or breast cancer patients. The method comprises: contacting the sample with a product targeting the cell surface or intracellular biomarker on the pancreatic cancer and/or breast cancer circulating tumor cells (CTCs) to capture the circulating tumor cells, wherein the product is fixed on the surface of a solid support.

The present disclosure has the following beneficial effects:

In view of the limitations of existing products for CTC capture, the present disclosure screens for potential surface biomarkers of CTCs in the blood of pancreatic cancer and breast cancer patients by performing single-cell sequencing on CTC samples isolated from the blood of pancreatic cancer and breast cancer patients, and comparing and analyzing the sequencing results based on the pancreatic cancer and breast cancer data from public databases. Through further bioinformatics analysis, a series of candidate surface biomarkers of CTCs (CD9, CD41, THBS1, RGS18, RGS10) of pancreatic cancer and breast cancer were identified. These biomarkers, alone or in combination with existing biomarkers (EPCAM), were subjected to a CTC capture experiment using a microfluidic chip coated with a corresponding antibody, in which CTCs in the pancreatic cancer and breast cancer cell lines and in the blood sample of patients were enriched and screened in vitro, and the reliability and effectiveness of the biomarkers were verified. In the present disclosure, by using antibodies against novel surface biomarkers and combinations thereof, the enrichment and detection rate of CTCs in the blood of pancreatic cancer and breast cancer patients are significantly improved, and the possibility of missed detection is effectively reduced, thus overcoming the defects of low detection rate and small detection number of CTCs in traditional technologies. This technology greatly improves the capture and enrichment efficiency of CTCs, thus providing strong support for dynamic tumor monitoring, efficacy evaluation, and personalized precise treatment of pancreatic cancer and breast cancer patients, offering broad market application potential and benefiting more cancer patients.

### BRIEF DESCRIPTION OF THE DRAWINGS

In order to more clearly illustrate the embodiments of the present disclosure or the technical solutions in the prior art, the drawings to be used in the description of the embodiments or the prior art will be briefly introduced below. Obviously, the drawings in the description below are only some embodiments of the present disclosure, and for a person of ordinary skill in the art, other drawings can be obtained based on these drawings without creative work.

FIG. 1 is a diagram showing the gene expression abundance distribution of surface biomarkers of circulating tumor cells.

FIG. 2 is a schematic flow chart showing the process of sample capture and collection, fluorescence observation, and single-cell transcriptome sequencing.

FIG. 3 is a picture of a CTC capture and collection device.

FIG. 4 shows images of the captured CTCs (the left panel shows an image with a scale of 100 m, and the right panel shows an enlarged image with a scale of 50 m).

FIG. 5 shows a diagram of the capture and enrichment efficiency data of CTCs in each group of microfluidic chips after the whole blood from the portal vein of a pancreatic cancer patient flows through a microfluidic chip on which an EPCAM antibody combined with one or more antibodies against RGS18, RSG10, CD41, CD9, and THBS1 are fixed, respectively.

FIG. 6 shows a diagram of the capture and enrichment efficiency data of CTCs in each group of microfluidic chips after the whole blood from the peripheral vein of a breast cancer patient flows through a microfluidic chip on which an EPCAM antibody combined with one or more antibodies against RGS18, RSG10, CD41, CD9, and THBS1 are fixed, respectively.

FIG. 7 shows a diagram of the capture and enrichment efficiency data of each group of microfluidic chips for five types of human pancreatic cancer cells after human pancreatic cancer cell lines, SU86.86, CFPAC-1, PANC-1, ASPC-1, and MIA PACA-2 cells, flow through a microfluidic chip on which an EPCAM antibody combined with one or more antibodies against RGS18, RSG10, CD41, CD9, and THBS1 are fixed, respectively.

FIG. 8 shows a diagram of the capture and enrichment efficiency data of each group of microfluidic chips for five types of human breast cancer cells after human breast cancer cell lines, MCF-7, SKBR-3, MCF-12A, MCF-10A, and MDA-MB-231 cells, flow through a microfluidic chip on which an EPCAM antibody combined with one or more antibodies against RGS18, RSG10, CD41, CD9, and THBS1 are fixed, respectively.

### DETAILED DESCRIPTION OF THE EMBODIMENTS

In the present disclosure, unless otherwise specified, scientific and technical terms used herein have the meanings commonly understood by those skilled in the art. In addition, the terms and laboratory procedures related to protein and nucleic acid chemistry, molecular biology, cell and tissue culture, microbiology, immunology used herein are terms and routine procedures widely used in the corresponding fields. Also, for a better understanding of the present disclosure, definitions and explanations of relevant terms are provided below.

As used herein, the terms “individual”, “patient”, or “subject” can be used interchangeably and refer to any single animal to be treated, more preferably a mammal (including non-human animals, such as cats, dogs, horses, rabbits, zoo animals, cows, pigs, sheep, and non-human primates). In certain embodiments, the patient herein is a human. The patient may be a “cancer patient”, i.e., a patient suffering from a cancer, or at a risk of developing a cancer, or having one or more symptoms of a cancer.

As used herein, the term “tumor” refers to all neoplastic cell growth and proliferation, whether malignant or benign, and all precancerous and cancerous cells and tissues.

As used herein, the terms “cancer” and “cancerous” refer to or describe physiological diseases typically characterized by unregulated cell growth in mammals.

As used herein, the terms “cancer”, “cancerous”, “cell proliferative disorder”, “proliferative disorder” and “tumor” are not mutually exclusive when mentioned herein.

As used herein, the term “disorder” refers to any condition that would benefit from treatment, including but not limited to chronic and acute disorders or diseases, including those pathological conditions that predispose mammals to the disorder in question.

As used herein, the term “tumor cell” refers to any tumor cell present in a tumor or a sample thereof. Methods known in the art and/or described herein may be used to distinguish tumor cells from other cells that may be present in a tumor sample, such as stromal cells and tumor-infiltrating immune cells.

As used herein, the term “circulating tumor cells” or “CTCs” refers to tumor cells that are shed from a tumor and present in the blood (i.e., in the circulation).

As used herein, the term “tumor cell surface biomarker” refers to such biological molecules, e.g., proteins, carbohydrates, glycoproteins, etc. These biological molecules are exclusively, preferentially or differentially expressed on tumor cells, and/or are found to be associated with tumor cells, and thus can serve as preferred or specific targets for tumors. In specific embodiments, dominant expression may be a dominant expression compared to other cells in an organism, or a dominant expression in a particular region (e.g., in a particular organ or tissue) of an organism.

As used herein, the term “sample” or “test sample” refers to a sample acquired or isolated from a biological organism, such as a tumor sample from a subject. Exemplary biological samples include, but not limited to: biofluid samples, serum, plasma, urine, saliva, tumor samples, tumor biopsies, and/or tissue samples, etc. This term also includes mixtures of the above samples. The term “test sample” also includes untreated or pretreated (or preprocessed) biological samples. In some embodiments, the test sample may include cells from a subject. In some embodiments, the test sample may be a tumor cell test sample, for example, the sample may include cancer cells, cells from a tumor and/or tumor biopsies. In some embodiments, the test sample may be a blood sample. The test sample can be obtained by removing cell samples from the subject, but can also be obtained from previously isolated cells (e.g., isolated at a previous time point or isolated by the same person or by another person). In addition, the test sample may be a freshly-collected or previously-collected sample.

As used herein, the term “detection” includes any detection means, including direct and indirect detection.

### Example 1: Screening and Identification of Surface Biomarkers of Circulating Tumor Cells

I. Single-Cell Transcriptome Sequencing of Circulating Tumor Cells from Clinical Samples

### 1. Collection of Clinical Samples and Isolation of CTCs

In order to find, screen and identify surface biomarkers of circulating tumor cells, blood from cancer patients was collected, from which CTCs were extracted. A microfluidic chip was used to capture CTCs from portal blood for single-cell transcriptome sequencing. The capture process includes the following steps: (1) before capturing the CTCs, injecting anti-EPCAM capture antibodies into the channels of a microfluidic chip and coating at 4° C. overnight to prepare a microfluidic capture system for capturing CTCs of pancreatic cancer; (2) on the day of capture, collecting blood sample from the patient, and removing red blood cells using red blood cell lysis buffer; (3) resuspending the cells with HBSS to a density of 2×10⁷ cells/mL, and injecting into the microfluidic chip at a flow rate of 5 mL/h; (4) flushing 3 times with 10 mL of HBSS at a flow rate of 30 mL/h to remove non-specifically bound cells; (5) flushing the chip with 15 mL of elution buffer at a flow rate of 50 mL/h to release and collect the CTCs. The collected CTCs can be used for single-cell transcriptome sequencing.

### 2. Construction and Sequencing of 10×Genomics Single-Cell Library

In the present disclosure, the 10×Genomics Chromium 3′ Gene Expression Kit V3 was used to prepare a single-cell transcriptome library for sequencing, and the detailed steps were strictly implemented in accordance with the 10×Genomics single-cell operation specification. Specifically, after obtaining a single-cell suspension after the above steps, the target cells and the corresponding 10×Genomics reagents were added to the Chromium chip to generate a Gel Bead in Emulsion (GEM) containing a single cell and a single gel bead, which was followed by subsequent reverse transcription, double-stranded DNA generation, library construction and sequencing. For the experiments, we set the target capture cells for each sample to be 6000 to 8000. The final constructed library was sequenced on the Illumina HiSeq 4000 platform, and the target sequencing depth for each cell was 100,000 reads.

### II. Identification of Surface Biomarkers of Circulating Tumor Cells (CTCs)

### 1. Single-Cell Transcriptome Data Preprocessing and Quality Control

First, the raw sequencing data images obtained from the above sequencing were converted into paired-end Fastq format sequences with 150 base pairs per read using the bcl2fastq software from Illumina Co. The obtained sequences were then aligned with the GRCh38 version of the human reference genome using Cell Ranger (v.3.0.0, 10×Genomics) software to obtain a gene expression matrix. The UMI tags of sequences that can be exclusively mapped to the exonic regions of transcriptomic genes will be used for subsequent statistical counting.

2. Copy Number Variation Analysis and Cell Type Identification, Along with Dimensionality Reduction and Clustering Analysis of Single-Cell Transcriptome Data

First, we performed global normalization of the gene expression matrix of single-cell data using the “LogNormalize” method. Specifically, the expression value of each gene was divided by the total expression of the corresponding cell and multiplied by a default parameter factor of 10,000, followed by a logarithmic transformation. Next, the “Find Variable Features” function was used to screen out 2,000 highly variable genes, and the “ScaleData” function was applied to scale the expression data of these genes to z-scores. Subsequently, principal component analysis (PCA) was performed on these highly variable genes using the “RunPCA” function to achieve dimensionality reduction of the data. After the dimensionality reduction was completed, a KNN graph was constructed using the “Find Neighbors” function to determine the weight relationship between cells. Cell clustering was performed by the “Find Clusters” function combined with the Louvain algorithm, with the resolution parameter set to 1. Finally, the clustering results were visualized using the t-SNE method.

### 3. Differential Gene Expression Analysis

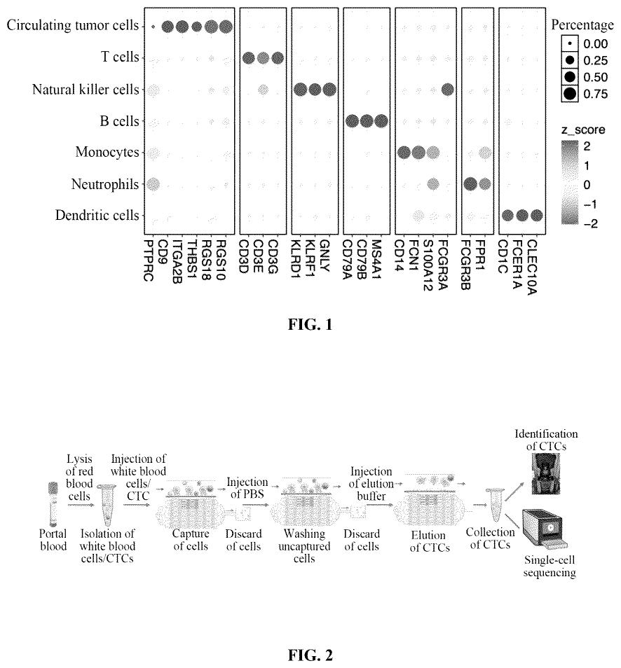

In order to find and identify differentially expressed genes in CTCs, the present disclosure used R studio to perform differential expression analysis through the “Find Markers” function, so as to evaluate the significance of genes based on the non-parametric Wilcoxon rank-sum test. The min.pct parameter was set to 0.25 and the logfc threshold parameter was set to 0.25. The surface biomarkers of circulating tumor cells were finally identified as CD9, CD41 (ITGA2B), THBS1, RGS18, RGS10 (as shown in FIG. 1), and T cells (CD3D+, CD3E+, CD3G+), natural killer cells (KLRD1+, KLRF1+, GNLY+), B cells (CD79A+, CD79B+, MS4A1+), monocytes (CD14+, FCN1+, S100A12+), neutrophils (FCGR3B+, FPR1+), dendritic cells (CD1C+, FCER1A+, CLEC10A+) were identified through analysis.

The relevant information of the biomarkers is shown in Tables 1 to 5.

TABLE 1

| | |
|---|---|
| Protein Name | Tetraspanin 9 |
| Gene Name | CD9 |
| Tissue-specific | Blood cells, immune organs (e.g., |
| | spleen and lymph gland), reproductive |
| | organs (e.g., ovary and testis), |
| | heart, liver, and lung |
| Cell type-specific | Immune cells, erythroid precursors |
| | and platelets, sperm cells and eggs, |
| | epithelial cells, tumor cell |
| Specificity in blood | Platelets, immune cells and erythroid |
| | precursors |
| Specificity in brain tissues | Low expression |
| Cancer prognosis correlation | Possibly related |
| Subcellular localization | Cytomembrane |

TABLE 2

| | |
|---|---|
| Protein Name | Regulatory G protein signal 18 |
| Gene Name | RGS18 |
| Tissue-specific | Platelets and hematopoietic system |
| Cell type-specific | Hematopoietic cells, platelets, |
| | megakaryocytes |
| Specificity in blood | High expression in platelets |
| Specificity in brain tissues | Low |
| Cancer prognosis correlation | Possibly related |
| Subcellular localization | Cytomembrane, cytoplasm |

TABLE 3

| | |
|---|---|
| Protein Name | Integrin alpha IIb chain |
| Gene Name | ITGA2B |
| Tissue-specific | Hematopoietic system |
| Cell type-specific | Platelets, megakaryocytes |
| Specificity in blood | High expression in platelets |
| Specificity in brain tissues | None |
| Cancer prognosis correlation | Possibly related |
| Subcellular localization | Cytomembrane |

TABLE 4

| | |
|---|---|
| Protein Name | Regulator of G-protein signaling 10 |
| Gene Name | RGS10 |
| Tissue-specific | Brain tissue, bone marrow, spleen |
| Cell type-specific | Immune cells, nerve cells, bone |
| | marrow cells |
| Specificity in blood | Platelets and some immune cells |
| Specificity in brain tissues | Cerebellum, hippocampus, and cortex |
| Cancer prognosis correlation | Possibly related |
| Subcellular localization | Cytomembrane, cytoplasm, cell nucleus |

TABLE 5

| | |
|---|---|
| Protein Name | Thrombospondin 1 |
| Gene Name | THBS1 |
| Tissue-specific | Multiple tissues |
| Cell type-specific | Vascular endothelial cells, smooth muscle |
| | cells, fibroblasts, platelets, immune |
| | cells, and epithelial cells |
| Specificity in blood | High expression in platelets |
| Specificity in brain tissues | Glial cells and neurons |
| Cancer prognosis correlation | High level of THBS1 related to poor |
| | prognosis |
| Subcellular localization | Cytomembrane |

### Example 2: Capture Capacity of Combined Antibodies for CTCs in the Blood of Pancreatic Cancer Patients

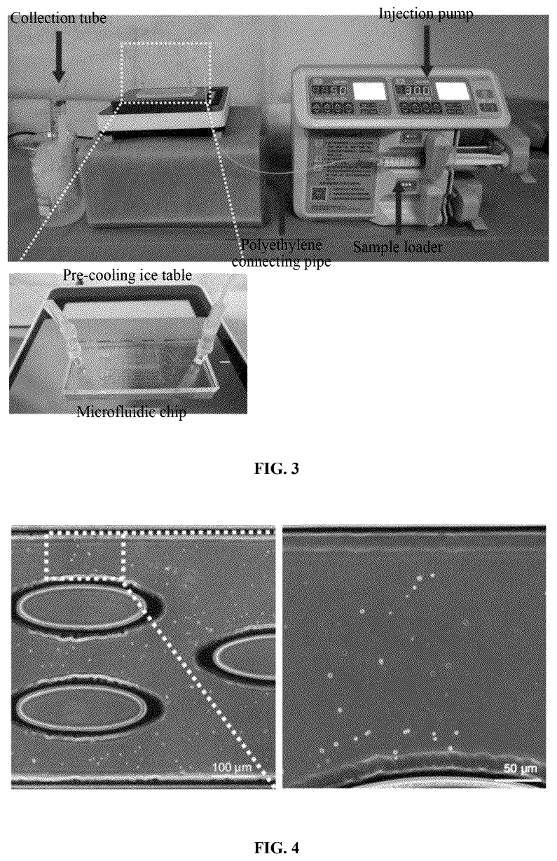

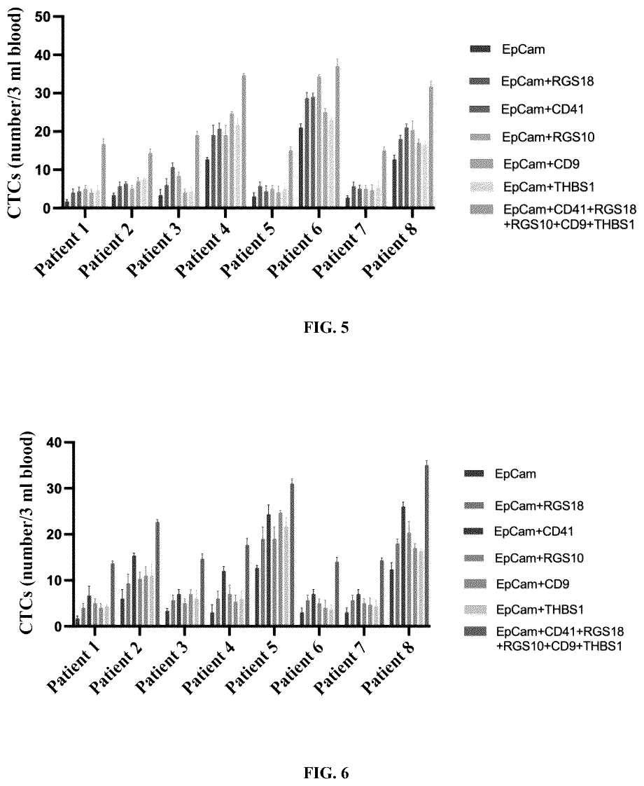

The CTCs in the blood of pancreatic cancer patients were captured and enriched using the screened cell surface biomarkers. The specific process was as shown in FIG. 2: (1) the combined antibodies against the surface biomarkers of CTCs (EPCAM, EPCAM and RGS18; EPCAM and CD41; EPCAM and RGS10; EPCAM and CD9; EPCAM and THBS1; EPCAM and RGS18, CD41, RGS10, CD9, THBS1) were injected into the channels of a microfluidic chip and coated at 4° C. overnight to prepare a microfluidic capture system for capturing CTCs of pancreatic cancer; (2) red blood cells were removed from the fresh blood sample collected from the patient using red blood cell lysis buffer; (3) CTCs were captured and collected from the resulting cell samples by using the CTC capture device as shown in FIG. 3. (4) The collected cells were stained with fluorescent staining reagents, and finally scanned and counted with a fluorescence counter to count the number of the captured CTCs (as shown in FIG. 5).

The results showed that the enrichment and screening capacities of the chips coated with combined antibodies against EPCAM and RGS18, EPCAM and CD41, EPCAM and RGS10, EPCAM and CD9, or EPCAM and THBS1 for CTCs in the blood sample of pancreatic cancer patients were significantly higher than those of the chips coated with EPCAM alone (as shown in FIG. 5), and the enrichment and screening capacities of the chips coated with combined antibodies against EPCAM and RGS18, CD41, RGS10, CD9, THBS1 for CTCs in the blood sample of pancreatic cancer patients were significantly higher than those of the chips coated with combined antibodies against EPCAM and RGS18, EPCAM and CD41, EPCAM and RGS10, EPCAM and CD9, or EPCAM and THBS1 (as shown in FIG. 5). The product device is shown in FIG. 3, mainly including a capture and collection system, which includes a microfluidic injection pump, a chip coated with antibody combinations against different surface biomarkers, and a collection device. The images of the captured CTCs were shown in FIG. 4.

The specific operation flow chart is shown in FIG. 2. The figure shows the workflow in which cells are extracted from the peripheral venous whole blood of a patient and passed through a microfluidic chip fixed with EPCAM and new biomarker antibodies, and after being washed with a buffer solution and eluted with a washing solution, tumor cells are enriched and subsequently subjected to fluorescence staining, observation, and single-cell transcriptome sequencing.

### Example 3: Capture Capacity of Combined Antibodies for CTCs in the Blood of Breast Cancer Patients

The CTCs in the blood of breast cancer patients were captured and enriched using the screened cell surface biomarkers. The specific process was as shown in FIG. 2: (1) the combined antibodies against the surface biomarkers of CTCs (EPCAM, EPCAM and RGS18; EPCAM and CD41; EPCAM and RGS10; EPCAM and CD9; EPCAM and THBS1; EPCAM and RGS18, CD41, RGS10, CD9, THBS1) were injected into the channels of a microfluidic chip and coated at 4° C. overnight to prepare a microfluidic capture system for capturing CTCs of breast cancer; (2) red blood cells were removed from the fresh blood sample collected from the patient using red blood cell lysis buffer; (3) CTCs were captured and collected from the resulting cell samples by using the CTC capture device as shown in FIG. 3. (4) The collected cells were stained with fluorescent staining reagents, and finally scanned and counted with a fluorescence counter to count the number of the captured CTCs (as shown in FIG. 6).

The results showed that the enrichment and screening capacities of the chips coated with combined antibodies against EPCAM and RGS18, EPCAM and CD41, EPCAM and RGS10, EPCAM and CD9, or EPCAM and THBS1 for CTCs in the blood sample of breast cancer patients were significantly higher than those of the chips coated with EPCAM alone (as shown in FIG. 6), and the enrichment and screening capacities of the chips coated with combined antibodies against EPCAM and RGS18, CD41, RGS10, CD9, THBS1 for CTCs in the blood sample of breast cancer patients were significantly higher than those of the chips coated with combined antibodies against EPCAM and RGS18, EPCAM and CD41, EPCAM and RGS10, EPCAM and CD9, or EPCAM and THBS1 (as shown in FIG. 6). The product device is shown in FIG. 3, mainly including a capture and collection system, which includes a microfluidic injection pump, a chip coated with antibody combinations against different surface biomarkers, and a collection device.

The specific operation flow chart is shown in FIG. 2. The figure shows the workflow in which cells are extracted from the peripheral venous whole blood of a patient and passed through a microfluidic chip fixed with EPCAM and new biomarker antibodies, and after being washed with a buffer solution and eluted with a washing solution, tumor cells are enriched and subsequently subjected to fluorescence staining, observation, and single-cell transcriptome sequencing.

### Example 4: Capture Efficiency of Combined Antibodies for Different Pancreatic Cancer Cells

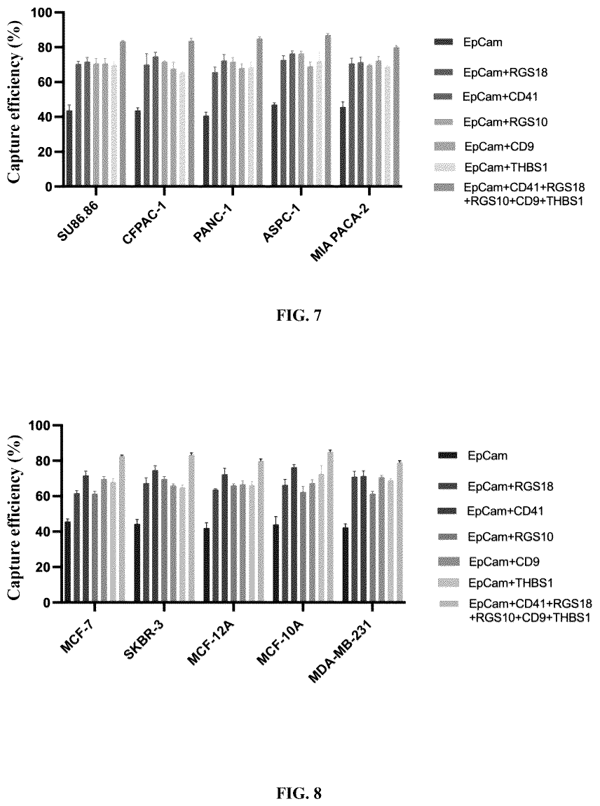

Human pancreatic cancer cell lines (SU86.86, CFPAC-1, PANC-1, ASPC-1, MIA PACA-2 cells) were flowed through microfluidic chips fixed with an EPCAM antibody alone or in combination with one or more antibodies against RGS18, RSG10, CD41, CD9, and THBS1 at a certain rate, with 1000 cells each group. The cells captured by the chips were collected using a collection solution and counted, so as to calculate the capture capacity of different antibodies for five types of cells, SU86.86, CFPAC-1, PANC-1, ASPC-1, MIA PACA-2 (as shown in FIG. 7).

Human pancreatic cancer cells SU86.86, CFPAC-1, PANC-1, ASPC-1, MIA PACA-2 were captured using chips coated with an EPCAM antibody alone or in combination with one or more antibodies against RGS18, RSG10, CD41, CD9, and THBS1, and the percentage of the captured and enriched cells were tested (inputting the total number of cells). As can be seen from FIG. 7, the capture efficiency of EPCAM in combination with RGS18, RSG10, CD41, CD9, and THBS1 antibodies was significantly higher than that obtained when EPCAM was used alone, and the capture capacity of the chip coated with combined antibodies against EPCAM and RGS18, CD41, RGS10, CD9, and THBS1 for human pancreatic cancer cells was significantly higher than those of chips coated with combined antibodies against EPCAM and RGS18, EPCAM and CD41, EPCAM and RGS10, EPCAM and CD9, or EPCAM and THBS1 (as shown in FIG. 7).

### Example 5: Capture Efficiency of Combined Antibodies for Different Breast Cancer Cells in the Blood

Human breast cancer cell lines (MCF-7, SKBR-3, MCF-12A, MCF-10A, MDA-MB-231 cells) were flowed through microfluidic chips fixed with an EPCAM antibody alone or in combination with one or more antibodies against RGS18, RSG10, CD41, CD9, and THBS1 at a certain rate, with 1000 cells each group. The cells captured by the chips were collected using a collection solution and counted, so as to calculate the capture capacity of different antibodies for five types of cells, MCF-7, SKBR-3, MDA-MB-231, MCF-12A, MCF-10A (as shown in FIG. 8).

Human breast cancer cells MCF-7, SKBR-3, MDA-MB-231, MCF-12A, MCF-10A were captured using chips coated with an EPCAM antibody alone or in combination with one or more antibodies against RGS18, RSG10, CD41, CD9, and THBS1, and the percentage of the captured and enriched cells were tested (inputting the total number of cells). As can be seen from FIG. 8, the capture efficiency of EPCAM in combination with RGS18, RSG10, CD41, CD9, and THBS1 antibodies was significantly higher than that obtained when EPCAM was used alone, and the capture capacity of the chip coated with combined antibodies against EPCAM and RGS18, CD41, RGS10, CD9, and THBS1 for human breast cancer cells was significantly higher than those of chips coated with combined antibodies against EPCAM and RGS18, EPCAM and CD41, EPCAM and RGS10, EPCAM and CD9, or EPCAM and THBS1 (as shown in FIG. 8).

The foregoing is only a specific embodiment of the present disclosure, but the scope of protection of the present disclosure is not limited thereto, and any skilled person familiar with the technical field can easily think of changes or substitutions within the technical scope as disclosed in the present disclosure, which shall be covered by the scope of protection of the present disclosure. Therefore, the scope of protection of the present disclosure shall be subject to the scope of protection of the claims.

What is claimed is:

1. Use of a biomarker panel in the preparation of a detection kit for capturing circulating tumor cells, wherein the biomarker panel comprises EPCAM, CD9, CD41, THBS1, RGS18, and RGS10;
the circulating tumor cells are pancreatic cancer circulating tumor cells,
and/or, the circulating tumor cells are breast cancer circulating tumor cells.

2. The use according to claim 1, wherein the detection kit comprises a detection reagent for detecting the biomarker panel, and the biomarker panel comprises EPCAM, CD9, CD41, THBS1, RGS18, and RGS10.

3. The use according to claim 1, wherein, the detection kit further comprises detection antibodies for corresponding biomarkers, a microfluidic chip, a buffer and a flushing fluid; wherein:
the detection antibodies are antibodies against CD9, CD41, THBS1, RGS18, RGS10, and EPCAM;
the microfluidic chip consists of a substrate layer containing multiple channels and a cover layer;
the buffer is a balanced salt solution containing 0.1 wt % BSA and 5 wt % glucose components; and
the flushing fluid is a balanced salt solution containing 0.1 wt % BSA, 0.2 wt % pancreatin, and 0.5 mg/mL papain.

4. Use of a biomarker panel in the preparation of a device for capturing circulating tumor cells, wherein, the biomarker panel comprises EPCAM, CD9, CD41, THBS1, RGS18, and RGS10.

5. The use according to claim 4, wherein, the device comprises a microfluidic chip, an injection pump, a connecting pipe, a pre-cooling ice table, and a sample collection tube; wherein:
the microfluidic chip is used to detect the biomarker panel on the surface of circulating tumor cells in a sample and capture the biomarker panel; the biomarker panel comprises EPCAM, CD9, CD41, THBS1, RGS18, and RGS10;
the injection pump is used to inject the sample into the microfluidic chip;
the connecting pipe is used to connect the injection pump, the microfluidic chip and the sample collection tube;
the pre-cooling ice table is used to keep the microfluidic chip at low temperature; and
the sample collection tube is used to collect the captured CTCs.

6. The use according to claim 5, wherein, the sample is a human blood sample.
